(19) United States
(12) Patent Application Publication
(10) Pub. No.: US 2003/0157048 A1
(43) Pub. Date: Aug. 21, 2003

(54) Pre-shampoo conditioning composition

(75) Inventors: Natsumi Komure, Ashiya-shi (JP); Ulpiano Bonifacio Dulguime II, Kobe (JP); Michael Albert Snyder, Mason, OH (US); Miwa Nagao, Nishinomiya-shi (JP); Yumi Kasakawa, Nishinomiya-shi (JP); Naohiro Morita, Kobe (JP); Kiro Harada, Kobe (JP); Louise Gail Scott, Horsell (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/375,595

(22) Filed: Feb. 27, 2003

Related U.S. Application Data
(63) Continuation of application No. PCT/US01/05739, filed on Feb. 23, 2001.

(51) Int. Cl. A61K 7/06; A61K 7/11

(52) U.S. Cl. 424/70.13

(57) ABSTRACT

Disclosed is a hair conditioning composition for applying to the dry hair prior to shampooing, comprising: (a) a conditioning oil which is in liquid form at 25° C.; and (b) a nonionic surfactant in an amount which provides phase inversion when the composition is in contact with an abundant amount of water.

### CROSS REFERENCE TO RELATED APPLICATION

The application is a continuation of International application PCT/US01/05739 (Case AA543F) filed on Feb. 23, 2001, which claims priority to application PCT/US00/24018 (Case AA493F) filed on Aug. 31, 2000.

### FIELD OF THE INVENTION

The present invention relates to pre-shampoo conditioning compositions which deliver conditioning benefit to the hair by applying on dry hair and subsequently washing the hair with a shampoo. Specifically, the present invention relates to pre-shampoo conditioning compositions having a phase inversion property.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to alleviate these after-shampoo problems. These approaches range from post-shampoo application of hair conditioners such as leave-on and rinse-off products, to hair conditioning shampoos which attempt to both clean and condition the hair from a single product.

Although some consumers prefer the ease and convenience of a shampoo which includes conditioners, a substantial proportion of consumers prefer the more conventional conditioner formulations which are applied to the hair as a separate step from shampooing. Generally, there are two types of product forms that consumers prefer using. One form is the rinse off type conditioners, which would be applied during a bathing regimen subsequent to shampooing the hair, and typically be rinsed off from the hair. Another form is the leave on type conditioners, which would be applied to the hair in a dry or partially wet condition, typically as part of a styling regimen, and the hair would be left to dry.

The use on hair of oils which are liquid at room temperature, such as coconut oil, is known in the art. Such oils are difficult to effectively deliver to the hair. When such oils are contained in rinse off conditioning formulations and applied to the hair in a wet condition subsequent to shampooing, the oils would not deposit on and/or penetrate in the hair in an effective manner, and thus easily washed off in the rinsing process. When such oils are contained in leave on conditioning formulations and applied in a relatively dry condition and left to dry, the hair would be left with a sticky feel and a negative appearance. Thus, the conditioning benefit of oils were not achieved with the conventional method of use for such oils.

Some consumers with fine hair have a desire to volume-up their hair. The term “hair volume-up” as used herein is not equal to fly-away hair. Fly-away hair is due to the increased level of static, and represents volume increase of only very minor amount of the hair as a whole, and is not desirable. On the other hand, hair volume-up as used herein relates to increase of the bulk of the hair volume. Consumers having fine hair have the desire to achieve hair volume-up while controlling undesirable fly-away of the hair. Generally, hair conditioner products targeted for such consumers provided the volume-up or less volume-down benefit by decreasing the level of conditioning actives, including oils, included in the composition. This is thought to be emerging from the concept that conditioning actives weigh down the hair. Consequently, hair conditioner products targeted for consumers which desire volume-up generally had only compromised conditioning benefits.

Based on the foregoing, there remains a desire to provide hair conditioning compositions which provide manageability and volume up to the hair while also providing a smooth, soft, and moisturized feel to the hair. There also remains a desire to provide hair conditioning compositions which provide such benefits without giving a sticky feel or heavy feel during and after use, or negative appearance after use. There is also a desire to provide hair conditioning compositions which provide such benefits which have a clear transparent appearance.

None of the existing art provides all of the advantages and benefits of the present invention.

### SUMMARY

The present invention is directed to a hair conditioning composition for applying to the dry hair prior to shampooing, comprising:

(a) a conditioning oil which is in liquid form at 25° C.; and

(b) a nonionic surfactant in an amount which provides phase inversion when the composition is in contact with an abundant amount of water.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Conditioning Oil

The composition of the present invention comprises a conditioning oil which is in liquid form at 25° C., preferably up to about 99.9%, more preferably from about 30% to about 99% by weight of the composition.

The conditioning oils useful herein are those which deposit on the surface of the hair when it is dry. Preferably, the conditioning oils useful herein are those which can, to some extent, penetrate into the hair and thus not washed off after subsequent shampooing. Still preferably, the conditioning oils useful herein are those which can, after phase inversion, provide controlled coverage to the surface of the hair.

Conditioning oils useful herein include those extracted or derived from a natural resource which contain a variety of compounds, and synthetic oils consisting of more or less a single compound. While volatile conditioning oils are not precluded herein, such oils are either kept to a relatively high boiling point or to a low level so as not to dry the hair, scalp, and hands.

Useful conditioning oils herein include vegetable oils, animal oils, and oils from other natural resources, synthetic oils, and mixtures thereof. The oils are selected according to the characteristics desired for the conditioner formulations. It is preferred that a certain amount of oils having a molecular weight of less than about 1500 is contained. Without being bound by theory, it is believed that oils having such molecular weight have the ability to penetrate into the hair.

In one preferred embodiment, paraffin oil is used at a level of at least about 50% of the composition. Such high-paraffin oil composition is believed to provide a light fresh feel to the hair with controlled coverage to the hair.

Vegetable oils useful herein are canola oil, camellia oil, olive oil, sunflower seed oil, cottonseed oil, soybean oil, peanut oil, olive oil, palm oil, corn oil, rapeseed oil, sesame oil, safflower oil, coconut oil, palm kernel oil, avocado oil, macadamia nut oil, corn oil, persic oil, wheat germ oil, pasanqua oil, linseed oil, perillic oil, teaseed oil, kaya oil, rice bran oil, china paulownia oil, Japanese paulownia oil, jojoba oil, rice germ oil, and mixtures thereof. Particularly preferred are canola oil, camellia oil, olive oil, sunflower seed oil, and mixtures thereof.

Animal oils and oils from other natural resources useful herein are sardine oil, lard, tallow, turtle oil, eggyolk oil, mink oil, squalane, lanolin, liquid lanolin, liquid paraffin, vaseline, and mixtures thereof.

Commercially available oils of natural resource useful herein include: canola oil with tradename Canola Salad Oil available from Ajinomoto, and tradenames Cropure Almond, Cropure Apricot Kernel, Cropure Avocado, and Cropure Peanut available from Croda.

Fatty alcohols useful herein as a conditioning oil include those having from about 10 to about 30 carbon atoms, preferably from about 12 to about 22 carbon atoms, and more preferably from about 16 to about 22 carbon atoms. These fatty alcohols can be straight or branched chain alcohols, preferably unsaturated alcohols. Nonlimiting examples of these compounds include oleyl alcohol, palmitoleic alcohol, linoleyl alcohol, and recinoleyl alcohol.

Fatty acids useful herein as a conditioning oil include those having from about 10 to about 30 carbon atoms, preferably from about 12 to about 22 carbon atoms, and more preferably from about 16 to about 22 carbon atoms. These fatty acids can be straight or branched chain acids and are unsaturated. Suitable fatty acids include, for example, oleic acid, linoleic acid, linolenic acid, ethyl linolenic acid, ethyl linolenic acid, arachidonic acid, and ricinolic acid.

Fatty acid derivatives and fatty alcohol derivatives useful herein as a conditioning oil are defined herein to include, for example, esters of fatty alcohols, alkoxylated fatty alcohols, alkyl ethers of fatty alcohols, alkyl ethers of alkoxylated fatty alcohols, and bulky ester oils such as pentaerythritol ester oils, trimethylol ester oils, citrate ester oils, glyceryl ester oils, and mixtures thereof. Nonlimiting examples of fatty acid derivatives and fatty alcohol derivatives, include, for example, methyl linoleate, ethyl linoleate, isopropyl linoleate, isodecyl oleate, isopropyl oleate, ethyl oleate, octyldodecyl oleate, oleyl oleate, decyl oleate, butyl oleate, methyl oleate, octyldodecyl stearate, octyidodecyl isostearate, octyidodecyl isopalmitate, octyl isopelargonate, octyl pelargonate, hexyl isostearate, isopropyl isostearate, isodecyl isononanoate, isopropyl isostearate, ethyl isostearate, methyl isostearate and Oleth-2. Bulky ester oils such as pentaerythritol ester oils, trimethylol ester oils, citrate ester oils and glyceryl ester oils useful herein are those which have a molecular weight of less than about 800, preferably less than about 500.

Hydrocarbons useful herein as a conditioning oil include straight chain, cyclic, and branched chain hydrocarbons which can be either saturated or unsaturated, so long as they have a melting point of not more than about 25° C. These hydrocarbons have from about 12 to about 40 carbon atoms, preferably from about 12 to about 30 carbon atoms, and more preferably from about 12 to about 22 carbon atoms. Also encompassed herein are polymeric hydrocarbons of alkenyl monomers, such as polymers of C₂₋₆ alkenyl monomers. These polymers can be straight or branched chain polymers. The straight chain polymers will typically be relatively short in length, having a total number of carbon atoms as described above. The branched chain polymers can have substantially higher chain lengths. The number average molecular weight of such materials can vary widely, but will typically be up to about 500, preferably from about 200 to about 400, and more preferably from about 300 to about 350. Also useful herein are the various grades of mineral oils. Mineral oils are liquid mixtures of hydrocarbons that are obtained from petroleum. Specific examples of suitable hydrocarbon materials include paraffin oil, mineral oil, dodecane, isododecane, hexadecane, isohexadecane, eicosene, isoeicosene, tridecane, tetradecane, polybutene, polyisobutene, and mixtures thereof.

Commercially available fatty alcohols and their derivatives useful herein include: oleyl alcohol with tradename UNJECOL 90BHR available from Shin Nihon Rika, various liquid esters with tradenames SCHERCEMOL series available from Scher, and hexyl isostearate with a tradename HIS and isopropryl isostearate having a tradename ZPIS available from Kokyu Alcohol. Commercially available hydrocarbons useful herein include mixtures of isoparaffins and cycloparaffins with molecular weight of about 330 with tradename CARNATION available from Witco, isododecane, isohexadeance, and isoeicosene with tradenames PERMETHYL 99A, PERMETHYL 101 A, and PERMETHYL 1082 available from Presperse (South Plainfield N.J., USA), a copolymer of isobutene and normal butene with tradenames INDOPOL H-100 available from Amoco Chemicals (Chicago Ill., USA), mineral oil with tradename BENOL available from Witco, BRITOL series and KAYDOL available from Witco, DRAKEOL series available from Penreco, MARCOL series available from Esso, and SUPERLA MINERAL OIL series available from Amoco Lubricants, and isoparaffin with tradename ISOPAR from Exxon Chemical Co. (Houston Tex., USA).

Poly α-olefin oils useful herein are those derived from 1-alkene monomers having from about 6 to about 16 carbons, preferably from about 6 to about 12 carbons atoms. Nonlimiting examples of 1-alkene monomers useful for preparing the poly α-olefin oils include 1-hexene, 1-octene, 1-decene, 1-dodecene, 1-tetradecene, 1-hexadecene, branched isomers such as 4-methyl-1-pentene, and mixtures thereof. Preferred 1-alkene monomers useful for preparing the poly α-olefin oils are 1-octene, 1-decene, 1-dodecene, 1-tetradecene, 1-hexadecene, and mixtures thereof. Poly α-olefin oils useful herein further have a viscosity of from about 1 to about 35,000 cst, a molecular weight of from about 200 to about 60,000, preferably less than about 6,000, and more preferably less than about 800; and a polydispersity of no more than about 3.

Commercialy available poly α-olefin oils herein include polydecenes with tradenames PURESYN 6 having a number average molecular weight of about 500 available from Mobil Chemical Co.

High molecular weight ester oils useful herein as a conditioning oil include pentaerythritol ester oils, trimethylol ester oils, citrate ester oils, glyceryl ester oils, and mixtures thereof. The high molecular weight ester oils herein are “water-insoluble”. As used herein, the term “water-insoluble” means the compound is substantially not soluble in water at 25° C.; when the compound is mixed with water at a concentration by weight of above 1.0%, preferably at above 0.5%, the compound is temporarily dispersed to form an unstable colloid in water, then is quickly separated from water into two phases.

Pentaerythritol ester oils useful herein are those having the following formula:

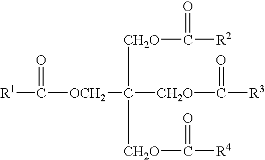

wherein R¹, R², R³, and R⁴, independently, are branched, straight, saturated, or unsaturated alkyl, aryl, and alkylaryl groups having from 1 to about 30 carbons. Preferably, R¹, R², R³, and R⁴, independently, are branched, straight, saturated, or unsaturated alkyl groups having from about 8 to about 22 carbons. More preferably, R¹, R², R³ and R⁴ are defined so that the molecular weight of the compound is from about 500 to about 1200.

Trimethylol ester oils useful herein are those having the following formula:

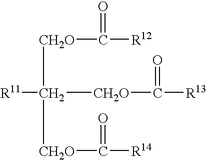

wherein R¹¹ is an alkyl group having from 1 to about 30 carbons, and R , R , and R independently, are branched, straight, saturated, or unsaturated alkyl, aryl, and alkylaryl groups having from 1 to about 30 carbons. Preferably, R¹¹ is ethyl and R ², R¹³, and R¹⁴, independently, are branched, straight, saturated, or unsaturated alkyl groups having from 8 to about 22 carbons. More preferably, R¹¹, R¹², R¹³ and R¹⁴ are defined so that the molecular weight of the compound is from about 500 to about 1200.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Citrate ester oils useful herein are those having a molecular weight of at least about 500 having the following formula:

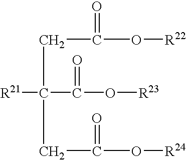

wherein R²¹ is OH or CH₃COO, and R²², R²³, and R²⁴, independently, are branched, straight, saturated, or unsaturated alkyl, aryl, and alkylaryl groups having from 1 to about 30 carbons. Preferably, R²¹ is OH, and R²², R²³, and R²⁴, independently, are branched, straight, saturated, or unsaturated alkyl, aryl, and alkylaryl groups having from 8 to about 22 carbons.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Glyceryl ester oils useful herein are those having a molecular weight of at least about 500 and having the following formula:

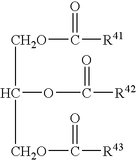

wherein R⁴¹, R⁴², and R⁴³, independently, are branched, straight, saturated, or unsaturated alkyl, aryl, and alkylaryl groups having from 1 to about 30 carbons. Preferably, R⁴¹, R⁴², and R⁴³, independently, are branched, straight, saturated, or unsaturated alkyl, aryl, and alkylaryl groups having from 8 to about 22 carbons.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Nonionic Surfactants

The composition of the present invention comprise nonionic surfactant in an amount which provides phase inversion when the composition is in contact with an abundant amount of water. Water may or may not be present in the composition, however, prior to contacting with an abundant amount of water, the composition of the present invention has an oil-continuous phase. Depending on the type and amount of nonionic surfactant used, water may be present as a co-solvent for the nonionic surfactant and aid phase inversion. A combination of surfactants can be used. When the composition is applied to dry hair and rinsed with water, phase inversion occurs to make a water-continuous phase, typically an oil-in-water phase. The type and amount of nonionic surfactant included in the present composition is controlled to provide such phase inversion, yet not to foam and wash the hair upon application at a dry condition.

Nonionic surfactants include those compounds produced by condensation of alkylene oxide groups, hydrophilic in nature, with an organic hydrophobic compound, which may be aliphatic or alkyl aromatic in nature.

Preferred nonlimiting examples of nonionic surfactants for use in the compositions include the following:

(1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the said ethylene oxide being present in amounts equal to from about 10 to about 60 moles of ethylene oxide per mole of alkyl phenol;

(2) those derived from the condensation of ethylene oxide with the product resulting from the reaction of propylene oxide and ethylene diamine products;

(3) condensation products of aliphatic alcohols having from about 8 to about 18 carbon atoms, in either straight chain or branched chain configurations, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms;

(4) long chain tertiary amine oxides of the formula [R¹ R²R³N→O] where R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals;

(5) long chain tertiary phosphine oxides of the formula [RR′R″→O] where R contains an alkyl, alkenyl or monohydroxyalkyl radical ranging from about 8 to about 18 carbon atoms in chain length, from 0 to about 10 ethylene oxide moieties and from 0 to 1 glyceryl moieties and R′ and R″ are each alkyl or monohydroxyalkyl groups containing from about 1 to about 3 carbon atoms;

(6) long chain dialkyl sulfoxides containing one short chain alkyl or hydroxy alkyl radical of from 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to 1 glyceryl moieties;

(7) alkyl polysaccharide (APS) surfactants (e.g. alkyl polyglycosides), examples of which are described in U.S. Pat. No. 4,565,647, which is incorporated herein by reference in its entirety, and which discloses APS surfactants having a hydrophobic group with about 6 to about 30 carbon atoms and a polysaccharide (e.g., polyglycoside) as the hydrophilic group; optionally, there can be a polyalkylene-oxide group joining the hydrophobic and hydrophilic moieties; and the alkyl group (i.e., the hydrophobic moiety) can be saturated or unsaturated, branched or unbranched, and unsubstituted or substituted (e.g., with hydroxy or cyclic rings); a preferred material is alkyl polyglucoside which is commercially available from Henkel, ICI Americas, and Seppic; and

(8) polyoxyethylene alkyl ethers such as those of the formula RO(CH₂CH₂)ₙH and polyethylene glycol (PEG) glyceryl fatty esters, such as those of the formula R(O)OCH₂CH(OH)CH₂(OCH₂CH₂)ₙOH, wherein n is from 1 to about 200, preferably from about 20 to about 100, and R is an alkyl having from about 8 to about 22 carbon atoms.

(9) other hydrophilic surfactants such as alkyl glucosides, polyglycerin fatty acid esters, polyglycerin alkyl ethers, polyoxyethylene sorbitan fatty acid esters, polyoxypropylene alkyl ethers, polyoxyethylene alkyl phenyl ethers, polyethylene glycol fatty acid esters, ethylene oxide derivatives of polypropylene glycol fatty acid esters, trimethylpropane fatty acid esters, polyoxyethylene hardened castor oil fatty acid esters, polyoxyethylene glyceryl pyroglutamate esters, and mixtures thereof.

Commercially available nonionic surfactants useful herein include C12-13 fatty alcohols with an average of 7 moles of ethylene oxide with tradenames Neodol 23-6.5 available from Shell and Dobanol 23-6.5 available from Mitsubishi, and Elfapur LT 65 SLN available from Akzo, sorbeth-40 tetraoleate with tradename Nikkol GO-440 available from Nikko Chemicals, sorbitan isostearate with tradename Crill 6 by Croda, Arlacel 987 available from ICI Americas, Montane-70 available from Seppic, and Nikkol SI-10T available from Nikko Chemicals, oleth-5 with tradename Volpo 5 available from Croda, Brox OL-5 available from Brooks, Ethoxol-5 available from Lanaetex, and Eumulgin 05 available from Henkel.

In one preferred embodiment, a small amount of water can be included in the composition. Without being bound by theory, the small amount of water is believed to help stabilize the surfactants in the composition, and further help the phase inversion upon contact of the composition with an abundant amount of water. Surprisingly, for products having only a small amount of or no thickener, the use of a small amount of water provides a clear transparent appearance to the composition. Clarity and/or transparency is an aesthetic benefit which may attract certain consumers who believe such appearance connote purity and mildness.

The amount of water should be kept to a level such that the composition is not completely emulsified. Preferably, water is kept to a level of no more than about 0.5%, more preferably from about 0.1% to about 0.5%.

Thickener

The composition of the present invention may further contain a thickener in an amount which provides the composition to have a viscosity of from about 100 mPa·s to about 200,000 mPa·s, preferably from about 500 mPa·s to about 100,000 mPa·s, more preferably from about 1,000 mPa·s to about 100,000 mPa·s. Such viscosity is suitable for providing a product which has a shear thinning property, good spreadability onto the hair, and thus providing improved deposition of the conditioning oils onto the hair, while not being too sticky. In one preferred embodiment, a thickener which provides a viscosity of from about 1,000 mPa·s to about 100,000 mPa·s is used for particularly good spreadability.

The viscosity herein is measured by a Brookfield DV-II+ viscometer. When the viscosity is above about 10,000 mPa·s, a #52 spindle is used and measured at a condition of using 0.5 ml of the sample at 1 rpm after 1 minute at 27° C. When the viscosity is below about 10,000 mPa·s, a #41 spindle is used and measured at a condition of using 2 ml of the sample at 1 rpm after 3 minutes at 27° C.

The thickeners useful herein are selected from the group consisting of fatty compounds, waxy compounds, gelling agents, inorganic thickeners, oil soluble polymers, and mixtures thereof. The amount and type of thickeners are selected according to the desired viscosity and characteristics of the product. A combination of thickeners can be used. One preferred combination is the use of a sucrose polyester and a 12-hydroxystearic acid or its esters. Preferably, the total amount of thickeners used is from about 0.1% to about 60%.

In one preferred embodiment, a thickener comprising a combination of from about 0.1% to about 10% fatty alcohol and from about 0.1% to about 50%, preferably up to about 30% of waxy compounds is used. In another preferred embodiment, from about 0.1% to about 10% of gelling agents is used. In another preferred embodiment, a thickener comprising from about 0.1% to about 10% inorganic thickeners is used.

Fatty Compounds

The fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that, depending on the number and position of double bonds, and length and position of the branches, certain compounds having certain required carbon atoms may have a melting point of less than 25° C. The fatty compounds useful as thickeners herein are only those having a melting point of 25° C. or higher. It is understood that the fatty compound thickeners herein may also provide conditioning benefits.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof. Fatty acid soaps are also useful herein. Nonlimiting examples of fatty acid soaps include natural soaps with Li, Na, Ca. Ba, and Al metals, including aluminium oleate and aluminium laurate.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

Commercially available materials useful herein include: myristyl myristate available from Croda with tradename Crodamol MM; cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy).

Fatty compounds useful herein include fatty acid sugar esters having C₁₋₃₀ monoester or polyester of sugars and one or more carboxylic acid moieties, preferably a sucrose polyester in which the degree of esterification is 7-8, and in which the fatty acid moieties are C₁₈ mono- and/or di-unsaturated and behenic, in a molar ratio of unsaturates:behenic of 1:7 to 3:5, more preferably the octaester of sucrose in which there are about 7 behenic fatty acid moieties and about 1 oleic acid moiety in the molecule, e.g., sucrose ester of cottonseed oil fatty acids. The thickening capability of these compounds are futher enhanced by adding about 1-3% of lower alcohols such as ethanol in the composition. Preferred compounds in this group include sucrose esterified with fatty acids derived from hardened, high-erucic acid rapeseed oil coded as SEFA behenate available from the Procter & Gamble Company.

Fatty compounds useful herein include aluminium salt of phophatididic acid, steroid derivatives, cholesterol esters, and Na, Li, K, and NH4 salts of 12-hydroxyoctadecanoic acid.

Waxy Compounds

Waxy compounds are useful as thickeners of the present invention. Useful herein are petrolatum, which is a mixture of hydrocarbons obtained from petroleum and which is semisolid at room temperature, paraffin wax, microcrystalline wax, ozokerite wax, ceresin wax, carnauba wax, candellila wax, eicosanyl behenate, and mixtures thereof. Petrolatum is particularly preferred. Commercially available waxy compounds useful herein include petrolatum having a tradename Super White Protopet available from Witco, Candelilla wax NC-1630 available from Noda wax, Ozokerite wax SP-1021 available from Strahl & Pitsh, and Eicosanyl behenate available from Cas Chemical.

Gelling Agents

The gelling agents useful as thickeners of the present invention include esters and amides of fatty acid gellants, hydroxy acids, hydroxy fatty acids, cholesterolic materials, lanolinolic materials, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:

R²—NH—CO—(CH₂)₂—CH—(NH—CO—R¹)—CO—NH—R²

wherein R¹ is an aliphatic hydrocarbon radical having from about 12 to about 22 carbon atoms, and R² is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercial available with tradename Gelling agent GP-1 available from Ajinomoto.

Amidoamines of the following general formula are useful herein:

R¹ CONH(CH₂)ₘN(R²)₂

wherein R¹ is a residue of C₁₁ to C₂₄ fatty acids, R² is a C₁ to C₄ alkyl, and m is an integer from 1 to 4. Preferred amidoamine useful in the present invention includes

| | |
|---|---|
| stearamidopropyldimethylamine, | stearamidopropyldiethylamine, |
| stearamidoethyldiethylamine, | stearamidoethyldimethylamine, |
| palmitamidopropyldimethylamine, | palmitamidopropyldiethylamine, |
| palmitamidoethyldiethylamine, | palmitamidoethyldimethylamine, |
| behenamidopropyldimethylamine, | behenamidopropyldiethylamine, |
| behenamidoethyldiethylamine, | behenamidoethyldimethylamine, |
| arachidamidopropyldimethylamine, | arachidamidopropyldiethylamine, |

arachidamidoethyldiethylamine, arachidamidoethyldimethylamine, and mixtures thereof; more preferably stearamidopropyldimethylamine, stearamidoethyldiethylamine, and mixtures thereof.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:

R¹—CO—(CH₂)₁₀—CH—(OH)—(CH₂)₅—CH₃

wherein R¹ is R² or NR²R³; and R² and R³ are hydrogen, or an alkyl, aryl, or arylalkyl radical which is branched linear or cyclic and has from about 1 to about 22 carbon atoms; preferably, from about 1 to about 18 carbon atoms. R² and R³ may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, triester of glycerin and hydroxystearic acid known as trihydroxystearin, and mixtures thereof; even more preferably, 12-hydroxystearic acid, isopropyl amide of 12-hydroxystearic acid, and combinations thereof.

Commercially available hydroxystearin compounds useful herein include 12-hydroxystearic acid (cosmetic grade) available from Kawaken and CasChem, and trihydroxystearin with tradenames Thixcin R available from Rheox, Flowtone R available from ECC America, and Rheocin available from United Catalysts.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, aspartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Other gellants useful herein include anthryl derivaties such as 2,3-bis n-decyloxyanthracene, hybrids of steroids and anthryl dervatives such as cholesterol anthraquinone-2-carboxylate, alpha amino acid oligomers such as N-benzyl oxycarbonyl-l-valyl-L-valine-n-octadecyl amide, organometallics such as mononuclear copper beta-diketonates and binulclear Cu and Rh tetracarboxylates, dextrin derivaties such as dextrin palmitate and dextrin myristate, and decaglycerin pentastearic acid.

Inorganic Thickeners

Inorganic thickeners useful herein include silica, oil soluble clays, and mixtures thereof. Highly dispersed, amorphous silicon dioxide of submicron particle size, also known as fumed silica, are particularly useful. Such material is commercially available as the Aerosil series (200, 300, 200CF, and 300CF) available from Degussa.

Oil Soluble Polymers

Oil soluble polymers are useful as thickeners of the present invention. Oil soluble polymers useful herein include guar gum which is a resinous material derived from the ground endosperm of cyanopsis tetragonoloba and close relatives.

Sensates

The hair conditioning composition of the present invention may further contain a sensate. As used herein the term “sensate” means a substance that, when applied to the skin, causes a perceived sensation of a change in conditions, for example, but not limited to, heating, cooling, refreshing and the like. Further, sensates may provide reduction of perceived oily and/or sticky feel during use.

Sensates are preferably utilized at levels of from about 0.001% to about 10%, more preferably from about 0.005% to about 5%, even more preferably from about 0.01% to about 1%, by weight, of the total compositions.

Any sensate suitable for use in hair care compositions may be used herein. Preferred sensates for use in the compositions herein are camphor, menthol, I-isopulegol, ethyl menthane carboxamide and trimethyl isopropyl butanamide.

Additional Components

A wide variety of other additional components can be formulated into the present compositions. These include: other conditioning agents such as Salcare SC96 which is a mixture of Polyquaternium 37, propylene glycol dicaprylate dicaprate, and PPG-1 trideceth-6 commercially available from Ciba Specialty Chemicals, hydrolysed collagen with tradename Peptein 2000 available from Hormel, vitamin E with tradename Emix-d available from Eisai, panthenol available from Roche, panthenyl ethyl ether available from Roche, a mixture of Polysorbate 60 and Cetearyl Alcohol with tradename Polawax NF available from Croda Chemicals, glycerylmonostearate available from Stepan Chemicals, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate, antidandruff agents such as zinc pyridinethione; and optical brighteners, for example polystyrylstilbenes, triazinstilbenes, hydroxycoumarins, aminocoumarins, triazoles, pyrazolines, oxazoles, pyrenes, porphyrins, imidazoles, and mixtures thereof.

Composition and Method of Use

The conditioning compositions of the present invention are for use to apply on dry hair prior to washing the hair with a shampoo. It has been surprisingly found that the conditioning compositions deliver conditioning benefit to the hair such as manageability and volume up to the hair when used in such way. The term “hair volume up” as used herein is not equal to fly-away hair. Fly-away hair is due to the increased level of static, and represents volume increase of only very minor amount of the hair as a whole, and is not desirable. On the other hand, hair volume up as used herein relates to increase of the bulk of the hair volume. Consumers having fine hair have the desire to achieve hair volume up while controlling undesirable fly-away of the hair. It has now been found that when applied to hair, a hair care composition as described herein may increase bulk hair volume, while reducing flyaway hair volume.

A suitable method of using the present composition comprises the steps of;

(a) applying the hair conditioning composition according to any of the claims above to dry hair; and

(b) shampooing the hair with a shampoo composition comprising a detersive surfactant.

By applying the present composition to the dry hair, it is believed that the oils deposit on the surface of the hair, or to some extent, penetrate into the hair, and thus stay on the hair even during the washing process of the hair. Due to the phase inversion properties of the present composition, if the composition were applied to wet hair, emulsification of the composition would occur before deposition and/or penetration to the hair. Accordingly, it is an element of the method of the present invention to apply the present composition to dry hair, rather than after the hair is wetted with water. Without being bound by theory, it is believed that, by treating the hair prior to shampooing and then washing the hair, soils on the hair are removed, while a certain amount of the conditioning oils of the present invention are left on the hair. The conditioning oil left in or on the hair is believed to provide significantly better manageability and appearance benefits to the hair compared to when the hair is washed without pretreatment of the hair with the present composition.

The shampoo composition to be used in step (b) can be any composition comprising detersive surfactants and is suitable for washing off soils from the hair. The term detersive surfactant, as used herein, is intended to distinguish these surfactants from surfactants which are primarily emulsifying surfactants, i.e. surfactants which provide an emulsifying benefit and which have low cleansing performance. It is recognized that most surfactants have both detersive and emulsifying properties. It is not intended to exclude emulsifying surfactants from the present invention, provided the surfactant also possesses sufficient detersive properties to be useful herein. Detersive surfactants are typically selected from the group consisting of anionic surfactants, amphoteric surfactants, nonionic surfactants, and mixtures thereof. The detersive surfactants comprised in the shampoo composition may or may not be the same as those comprised in the present conditioning composition. In one preferred embodiment, at least an anionic surfactant is included in the shampoo composition to be used in step (b).

The amount of the present composition used for treating the hair prior to shampooing depends on the volume of hair and desired conditioning benefits. Typically, from about 2 ml to about 40 ml, preferably from about 5 ml to about 20 ml of the present composition is used.

The present invention does not necessarily preclude overall hair treatment regimens wherein additional rinse off and/or leave on conditioning formulations are applied to the hair subsequent to washing the hair, or subsequent to washing and drying the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Examples 1 through 8 are hair conditioning compositions of the present invention which are used by applying to the dry hair prior to shampooing the hair.

Compositions of Examples 1 through 5

| Components | Ex. 1 | Ex. 2 | Ex. 3 | Ex. 4 | Ex. 5 |
|---|---|---|---|---|---|
| Paraffin Oil *1 | to make | to make | to make | to make | to make |
| | 100% | 100% | 100% | 100% | 100% |
| Canola Oil *2 | 20 | 20 | 20 | 20 | 20 |
| Sorbeth-40 | 4 | 4 | 4 | 4 | 4 |
| Tetraoleate *5 | | | | | |
| Sorbitan Isostearate *6 | 1 | 1 | 1 | 1 | 1 |
| Oleth-5 *7 | 4 | 4 | 4 | 4 | 4 |
| SEFA behenate *8 | | 5 | 1 | | |
| 12-Hydroxystearic | | | 1.5 | | |
| acid *9 | | | | | |
| Dibutyl Lauroyl | | | | 0.5 | |
| Glutamide *10 | | | | | |
| Trihydroxystearin *11 | | | | | 5 |
| p-Paraben | 0.1 | 0.1 | 0.1 | 0.1 | 0.1 |
| Vitamin E | 0.2 | 0.2 | 0.2 | 0.2 | 0.2 |
| Glycerine | 0.1 | 0.1 | 0.1 | 0.1 | 0.1 |
| DI Water | 0.3 | 0.3 | 0.3 | 0.3 | 0.3 |
| Perfume | 0.3 | 0.3 | 0.3 | 0.3 | 0.3 |
| Ethanol | | 3 | | | |

Compositions of Examples 6 through 8

| Components | Ex. 6 | Ex. 7 | Ex. 8 |
|---|---|---|---|
| Paraffin Oil *1 | to make 100% | to make 100% | to make 100% |
| Canola Oil *2 | 28 | 28 | 28 |
| Myristyl myristate *3 | 7 | 6 | 6 |
| C12-13 AE (6-7 | 4 | 4 | |
| EO) *4 | | | |
| Sorbeth-40 | | | 3 |
| Tetraoleate *5 | | | |
| Sorbitan Isostearate *6 | | | 1 |
| Oleth-5 *7 | | 1 | 0.5 |
| DI Water | 0.15 | 0.15 | 0.15 |
| Menthol | 0.1 | 0.5 | 0.1 |
| Perfume | 0.3 | 0.3 | 0.3 |
| Salcare SC96 *12 | 2 | | |

Definitions of Components
*1 Paraffin Oil: A blend of iso- and cyclo-paraffins with a molecular weight of approximately 330 with tradename Carnation available from Witco
*2 Canola Oil: Canola salad oil from Ajinomoto
*3 Myristyl myristate: Crodamol MM available from Croda
*4 C12-13 AE (6-7 EO): Neodol 23-6.5 available from Shell
*5 Sorbeth-40 Tetraoleate: Nikkol GO-440 available from Nikko Chemicals
*6 Sorbitan Isostearate: Crill 6 available from Nikko Chemicals
*7 Oleth-5: Volpo 5 available from Croda
*8 SEFA behenate: Sucrose esterified with long-chain fatty acids derived from hardened, high-erucic acid rapeseed oil available from the Procter & Gamble Company
*9 12-hydroxystearic acid: available from Kawaken
*10 Dibutyl Lauroyl Glutamide: GP-1 available from Ajinomoto
*11 Trihydroxystearin: Thixcin R available from Rheox
*12 Salcare SC96: Mixture of Polyquaternium 37, propylene glycol dicaprylate dicaprate, and PPG-1 trideceth-6 available from Ciba Specialty Chemicals

Preparation of Compositions

The compositions of Examples 1-8 as shown above can be prepared by any conventional method. A suitable method is described:

The paraffin oil is heated to above about 50° C., nonionic surfactants is added and stirred, and remaining conditioning oils are added. Preservatives and antioxidants, when present, can be premixed with one of the conditioning oils prior to mixing with other components. The remaining components except water and other heat sensitive components such as perfume and menthol are added. Finally, after the product has cooled down to about 35° C., if present, water, perfume, and menthol are added.

Example 2 is prepared by heating the paraffin oil to about 75° C., adding the SEFA behenate, and following the same method as described above. Ethanol is added after the product has cooled down to 35° C.

Example 3 is prepared by heating the paraffin oil to about 75° C., adding the 12-hydroxystearic acid and SEFA behenate, and following the same method as described above. The cooling of the composition from about 75° C. to about 30° C. is conducted in about 10 minutes.

Example 4 is prepared by heating the paraffin oil to about 160° C., adding Dibutyl Lauroyl Glutamide, and following the same method as described above.

Example 5 is prepared by heating the paraffin oil to about 80° C., adding the trihydroxystearin, and following the same method as described above.

The embodiments disclosed and represented by the previous examples have many advantages. For example, Examples 1 through 8 can provide conditioning benefit such as increase of bulk hair volume; body fullness; ease of combing, managing and styling the hair; smooth, soft, and moisturized feel to the hair, and shine to the hair. Examples 2 through 5 further have a viscosity of from about 1,000 mPa·s to about 100,000 mPa·s, and have good spreadability on the hair. Examples 1 and 6 through 8 further have a viscosity of at least 100 mPa·s, and have a clear appearance.

It is understood that the examples and embodiments described herein are for illustrative purposes only and that various modifications or changes in light thereof will be suggested to one skilled in the art without departing from its spirit and scope.

What is claimed is:

1. A hair conditioning composition for applying to dry hair prior to shampooing, comprising:
(a) a conditioning oil which is in liquid form at 25° C.; and
(b) a nonionic surfactant in an amount which provides phase inversion when the composition is in contact with an abundant amount of water.

2. The hair conditioning composition according to claim 1 further comprising a thickener.

3. The hair conditioning composition according to claim 2 wherein the thickener is a sucrose polyester.

4. The hair conditioning composition according to claim 2 wherein the thickener is a gelling agent selected from the group consisting of N-acyl amino acid amides, 12-hydroxystearic acid and its esters, and mixtures thereof.

5. The hair conditioning composition according to claim 2 wherein the thickener comprises a sucrose polyester and a 12-hydroxystearic acid or its esters.

6. The hair conditioning composition according to claim 1 further comprising a sensate.

7. The hair conditioning composition according to claim 1 wherein the conditioning oil comprises, by weight of the entire composition, at least about 50% of paraffin oil.

8. The hair conditioning composition according to claim 1 further comprising no more than about 0.5% water.

9. A method of conditioning the hair comprising the steps of;
(a) applying to dry hair a hair conditioning composition according to claim 1; and
(b) shampooing the hair with a shampoo composition comprising a detersive surfactant.

10. A method of increasing the bulk hair volume of the hair comprising the steps of;
(a) applying to dry hair a hair conditioning composition according to claim 1; and
(b) shampooing the hair with a shampoo composition comprising a detersive surfactant.

11. The method according to claim 7 wherein about 2 ml to about 40 ml of the hair conditioning composition is applied to the hair in step (a).
